(19) United States
(12) Patent Application Publication
(10) Pub. No.: US 2023/0080455 A1
(43) Pub. Date: Mar. 16, 2023

(54) GRID-BASED SOURCE-TRACING METHOD AND SYSTEM FOR SEWAGE OUTFALLS, AND STORAGE MEDIUM

(71) Applicant: TONGJI UNIVERSITY, Shanghai (CN)

(72) Inventors: Hailong YIN, Shanghai (CN); Yiyuan LIN, Shanghai (CN); Zuxin XU, Shanghai (CN)

(73) Assignee: TONGJI UNIVERSITY, Shanghai (CN)

(21) Appl. No.: 17/751,681

(22) Filed: May 24, 2022

Related U.S. Application Data
(63) Continuation-in-part of application No. PCT/CN2021/118627, filed on Sep. 16, 2021.

(51) Int. Cl. G01N 33/18; E03F 7/00; G01V 11/00

(52) U.S. Cl. CPC G01N 33/182; E03F 7/00; G01V 11/00

(57) ABSTRACT

A grid-based source-tracing method and system for sewage outfalls and a storage medium are provided. The method specifically includes the steps of: dividing a river into multiple reaches; determining monitoring sites according to the divided reaches; acquiring on-line monitoring data of each of the monitoring sites, and calculating soft measurement data; determining a river reach with sewage outfalls according to upstream and downstream soft measurement data; and intensively arranging monitoring sites in the river reach with sewage outfalls to subdivide the river reach with sewage outfalls, thereby determining a position of a sewage outfall. The method divides the river into multiple reaches and performs the grid-based source-tracing for the sewage outfall of the river gradually. In real practice, with online conductivity and water level monitoring data, the method can effectively determine the river reach with sewage outfalls using soft measurement.

### CROSS REFERENCE TO THE RELATED APPLICATIONS

This application is the continuation-in-part application of International Application No. PCT/CN2021/118627, filed on Sep. 16, 2021, the entire contents of which are incorporated herein by reference.

### TECHNICAL FIELD

The present disclosure relates to the technical field of source tracing for sewage discharge of rivers and, in particular, to a grid-based source-tracing method and system for sewage outfalls, and a storage medium.

### BACKGROUND

Investigation of sewage discharges into the river is the fundamental work in the river water quality restoration. The statistical sewage collection rate of urban areas in China at present has an average of more than 90%. However, measuring by the pollutant mass, the actual sewage collection rate only has an average of 60%, indicating that there are still lots of pollutants entering rivers. Sewage outfalls serve as the last “gates” for pollutants entering the rivers. The number of sewage outfalls and the pollutant discharge amount need to be clearly determined, which will improve practically the pollutant collection capacity and further improve the river water quality effectively.

The sewage outfalls are complicated. Especially great challenges are presented for underwater sewage outfalls. The conventional methods such as manual investigation and aerial survey of unmanned aerial vehicles (UAVs) are difficult to identify concealed underwater sewage outfalls. Underwater robots, thermal imagers and the like have been also put into use in recent years, but these have complicated operations, restrictions in the nighttime and other problems, which make the all-weather investigation be hardly implemented. Therefore, there is an urgent need for those skilled in the art to provide an investigation method and system for monitoring all sites in real time.

### SUMMARY

In view of this, the present disclosure provides a grid-based source-tracing method and system for sewage outfalls and a storage medium to monitor data of all positions in real time and to overcome the problems in the prior art.

To achieve the above objective, the present disclosure provides the following technical solutions:

A grid-based source-tracing method for sewage outfalls specifically includes the following steps:

dividing reaches: dividing a river into multiple reaches;

determining monitoring sites: determining the monitoring sites according to the divided reaches;

acquiring soft measurement data: acquiring on-line monitoring data of each of the monitoring sites, and calculating soft measurement data;

determining a river reach with sewage outfalls: determining the river reach with sewage outfalls according to upstream and downstream soft measurement data; and

obtaining a position of a sewage outlet: intensively arranging monitoring sites in the river reach with sewage outfalls to subdivide the river reach with sewage outfalls, thereby determining the position of the sewage outlet.

Optionally, when the monitoring sites are determined, a position for dividing the reaches and a confluence of a tributary may be determined as the monitoring sites.

Optionally, the acquiring monitoring data may include:

S31: acquiring a conductivity of each of the monitoring sites, thereby obtaining a chloride concentration of each of the monitoring sites according to a chloride concentration-conductivity curve; and

S32: synchronously acquiring a water level of each of the monitoring sites, thereby obtaining a flow of each of the monitoring sites according to a flow-water level curve.

Optionally, the chloride concentration-conductivity curve may be drawn as follows:

S311: acquiring a water sample from a fixed depth of each of the monitoring sites at a fixed frequency within a fixed time in a dry weather;

S312: measuring a conductivity and a chloride concentration of the acquired water sample; and

S313: performing, with a chloride concentration as a y axis and a conductivity as an x axis, linear fitting on the measured conductivity and chloride concentration with a least-squares method to obtain the chloride concentration-conductivity curve.

Optionally, the flow-water level curve may be drawn as follows:

S321: synchronously acquiring a flow and a water level of each of the monitoring sites at a fixed frequency within a fixed time; and

S322: performing, with a flow as an x axis and a water level as ay axis, polynomial fitting on the acquired flow and water level of each of the monitoring sites with the least-squares method to obtain the flow-water level curve.

Optionally, the river reach with sewage outfalls may be determined according to soft measurement data of upstream and downstream monitoring sites, where there are two cases, that is, there is a tributary and there is no tributary.

Optionally, in a case where a reach does not include a tributary, a river reach with sewage outfalls may be determined as follows:

determining variations of chloride concentrations of adjacent upstream and downstream monitoring sites:

determining, if Cᵢ>Cᵢ₋₁, that an i^{th} reach is the river reach with sewage outfalls;

where, i∈[1,n], Cᵢ is a daily averaged chloride concentration of an i^{th} monitoring site; Cᵢ₋₁ is a daily averaged chloride concentration of an upstream i−1^{th} monitoring site; and a 0^{th} monitoring site represents an upstream boundary of the river, namely C₀ is a daily averaged chloride concentration from an upstream inflow of the river; and

determining variations of chloride loads of adjacent upstream and downstream monitoring sites:

determining, if QᵢCᵢ>Qᵢ₋₁Cᵢ₋₁, that an i^{th} reach is the river reach with sewage outfalls,

where, i∈[1,n], Cᵢ is a daily averaged chloride concentration of an i^{th} monitoring site; Cᵢ₋₁ is a daily averaged chloride concentration of an upstream i−1^{th} monitoring site; Qᵢ is a daily flow of the i^{th} monitoring site; Qᵢ₋₁ is a daily flow of the upstream i−1^{th} monitoring site; and the 0^{th} monitoring site represents an upstream boundary of the river, namely C₀ is a daily averaged chloride concentration from an upstream inflow of the river, and Q₀ is a daily flow from the upstream inflow of the river.

Optionally, in a case where a reach includes a tributary, the river reach with sewage outfalls may be determined as follows:

comparing a chloride concentration of each of an upstream monitoring site, the tributary and a downstream monitoring site:

determining, if Cᵢ>max(Cᵢ₋₁,C_{Ti}), that an i^{th} reach is the river reach with sewage outfalls,

where, i∈[1,n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; and a 0^{th} monitoring site represents an upstream boundary of the river, namely C₀ is a daily averaged chloride concentration from an upstream inflow of the river; and

determining variations of chloride loads of adjacent upstream and downstream monitoring sites:

determining, if QᵢCᵢ>Qᵢ₋₁Cᵢ₋₁+Q_{Ti}C_{Ti}, then an i^{th} reach is the river reach with sewage outfalls,

where, i∈[1,n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; a 0^{th} monitoring site represents an upstream boundary of the river, namely C₀ is a daily averaged chloride concentration from an upstream inflow of the river; Qᵢ is a daily flow of the i^{th} monitoring site; Qᵢ₋₁ is a daily flow of the upstream i−1^{th} monitoring site; Q_{Ti} is a daily flow that the tributary flows into the i reach; and the 0^{th} monitoring site represents the upstream boundary of the river, namely the C₀ is the daily averaged chloride concentration from the upstream inflow of the river, and Q₀ is a daily flow from the upstream inflow of the river.

A grid-based source-tracing investigation system for sewage outfalls includes a data acquisition device, a data processing device, and a display device, where

the data acquisition device is configured to acquire tributary confluence data of a river, monitoring data of monitoring sites, and intensive monitoring data of a river reach with sewage outfalls;

the data processing device is configured to divide reaches according to the tributary confluence data of the river; calculate soft measurement data according to the monitoring data; determine the river reach with sewage outfalls according to the soft measurement data; and analyze the intensive monitoring data of the river reach with sewage outfalls to determine a position of a sewage outfall; and

the display device is configured to display the river reach with sewage outfalls and the position of the sewage outfall.

A computer storage medium stores a computer program thereon, where when executed by a processor, the program implements steps of the grid-based source-tracing method for sewage outfalls.

As can be seen from the above technical solutions, the grid-based source-tracing method and system for sewage outfalls, and a storage medium provided by the present disclosure achieve the following beneficial effects over the prior art:

(1) The present disclosure divides the river into multiple reaches and performs the grid-based source-tracing for sewage outfalls based on soft measurement. With online conductivity and water level monitoring data, the present disclosure can effectively determine the river reach with sewage outfalls. Moreover, the present disclosure has the accurate and convenient calculation method and solves the problem that the conventional methods such as manual investigation and aerial survey of UAVs difficultly identify concealed underwater sewage outfalls.

(2) The present disclosure selects the conservative substance, namely the chloride, as the water quality indicator. The chloride concentration is only affected by external loads and physical mixing with receiving water. Hence, the spatial distribution of the chloride concentrations can reflect input information of pollution sources to the greatest extent.

(3) The present disclosure constructs a soft measurement method for the chloride concentration and conductivity of the river. As the chloride concentration is positively related with the conductivity, the present disclosure converts the monitoring of the chloride concentration into the monitoring of the conductivity. By providing the online conductivity monitor, the present disclosure avoids sampling errors in water quality monitoring and is convenient in operation.

(4) The present disclosure constructs a soft measurement method for the water level and flow of the river and converts the monitoring of the flow into the monitoring of the water level, thereby solving problems of difficult flow monitoring and low measurement accuracy of the river, and being strongly practical.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description merely show the embodiments of the present disclosure, and those of ordinary skill in the art may still derive other drawings from the provided accompanying drawings without creative efforts.

FIG. 1 systematically illustrates a flow chart of a method according to the present disclosure;

FIG. 2 systematically illustrates a division of a river reach according to the present disclosure;

FIG. 3 illustrates a chloride concentration-conductivity curve according to an embodiment of the present disclosure;

FIG. 4 systematically illustrates a principle for monitoring a water flow of a section with a tracer-dilution method according to an embodiment of the present disclosure; and

FIG. 5 illustrates a flow-water level curve according to an embodiment of the present disclosure.

### DETAILED DESCRIPTION OF THE EMBODIMENTS

The technical solutions in the embodiments of the present disclosure are clearly and completely described below with reference to the accompanying drawings. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

Embodiments of the present disclosure provide a grid-based source-tracing method and system for sewage outfalls, and a storage medium, including the grid-based source-tracing method for sewage outfalls, the grid-based source-tracing investigation system for sewage outfalls, and the computer storage medium.

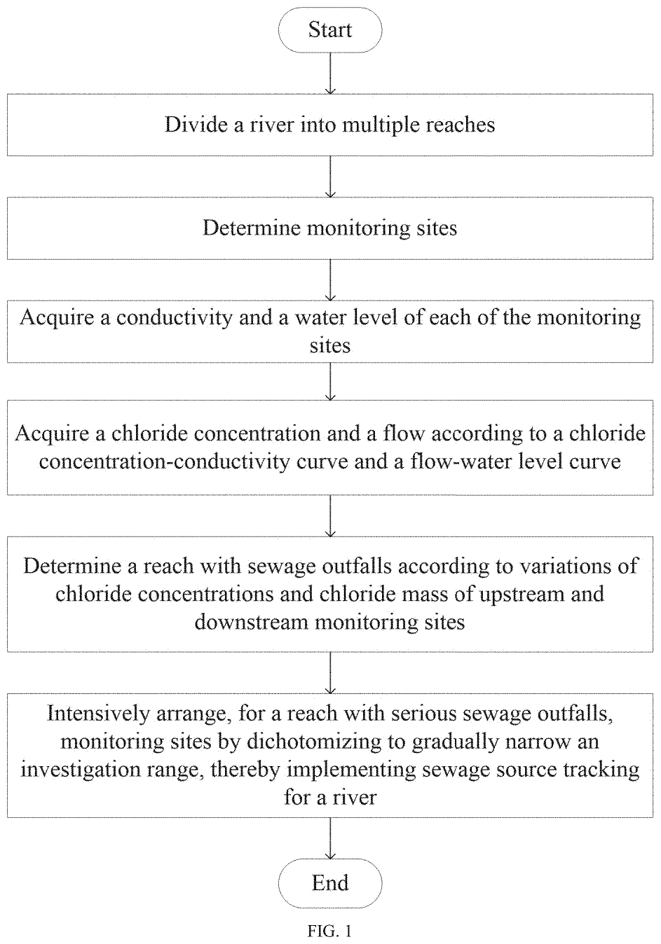

The grid-based source-tracing method for sewage outfalls specifically includes the following steps: dividing a river into multiple reaches and corresponding monitoring sites, providing an online water level and conductivity monitoring device at each of the monitoring sites, and conducting a grid-based investigation for a sewage outfall based on soft measurement; determining a river reach with sewage outfalls according to monitoring data of the reaches; and intensively arranging, for a reach with serious sewage discharge, monitoring sites to subdivide an investigation range, thereby implementing source tracing on the sewage outfall of the river. More specifically, as shown in FIG. 1, the grid-based source-tracing method includes the following steps:

A river is divided into n reaches, and a conductivity of each of monitoring sites is acquired, the monitoring sites being consistent with positions for dividing the reaches. A chloride concentration Cᵢ of each of the monitoring sites is acquired according to a corresponding chloride concentration-conductivity curve, i∈[1,n].

A water level of each of the monitoring sites is synchronously acquired, and a flow of each of the monitoring sites is acquired according to a corresponding flow-water level curve.

For a reach including a tributary, a conductivity and a water level of the tributary of the reach are monitored synchronously to obtain a water flow Q_{Ti} and a chloride concentration C_{Ti} of the tributary.

A river reach with sewage outfalls is determined according to variations of chloride concentrations and chloride loads of upstream and downstream monitoring sites.

For a reach with serious sewage discharge, monitoring sites are intensively arranged by dichotomizing to subdivide an investigation range, thereby implementing source tracing on the sewage outfalls of the river.

The chloride as a conservative substance is selected as the water quality monitoring indicator. As the chloride concentration is positively related with the conductivity, and the conductivity can be monitored online, monitoring of the chloride concentration is converted into monitoring of the conductivity based on soft measurement.

The chloride concentration-conductivity curve is specifically drawn as follows:

Water samples are acquired in the dry weather, the monitoring sites being consistent with the positions for dividing the reaches. The samples are continuously acquired once every 2 hours for 2-3 days. For each sampling point, it is required to acquire water samples at 0.5 m below the water surface. Following the acquisition of the water samples every day, they are sent to laboratories immediately to measure the conductivities and the chloride concentrations.

With a chloride concentration as a y axis and a conductivity as an x axis, linear fitting is performed on the monitoring data with a least-squares method to obtain the chloride concentration-conductivity curve.

As the water level is more easily monitored than the flow in the river, monitoring of the flow is converted into monitoring of the water level based on soft measurement.

The flow-water level curve is specifically drawn as follows:

A flow and a water level of each of the monitoring sites are synchronously acquired once every 4 h for 2-3 days.

With a flow as an x axis and a water level as ay axis, polynomial fitting is performed on the monitoring data with the least-squares method to obtain the flow-water level curve.

The flow is monitored with a tracer-dilution method, specifically including:

NaCl is selected as a tracer. A NaCl solution of a known concentration is instantly injected into an upstream station of the river, and water samples are continuously acquired at the downstream monitoring site until the tracer passes through the monitoring site completely. The conductivities of the water samples are monitored and converted into the chloride concentrations to obtain a time-varying curve of chlorides at the monitoring site. According to the chemical mass balance of the chlorides, the flow of the monitoring site is calculated by:

$$Q = \frac{M}{\sum ( {EC}_{t} - {EC}_{0} ) \cdot CF}$$

where, ECₜ is a conductivity when t=t, EC₀ is a background value for the conductivity of the downstream monitoring site, M is a mass of injected chlorides of an upstream site, and CF is a conversion coefficient between the conductivity and the chloride concentration, the value of the CF being obtained by referring to the chloride concentration-conductivity curve.

The river reach with sewage outfalls is determined according to the variations of the chloride concentrations and chloride loads of the upstream and downstream monitoring sites, which includes two cases:

First Case:

In a case where there is no tributary in a reach, a river reach with sewage outfalls is determined as follows:

Variations of chloride concentrations of adjacent upstream and downstream monitoring sites are determined:

An i^{th} reach is the river reach with sewage outfalls if Cᵢ>Cᵢ₋₁,

where, i∈[1,n], Cᵢ is a daily averaged chloride concentration of an i^{th} monitoring site; Cᵢ₋₁ is a daily averaged chloride concentration of an upstream i−1^{th} monitoring site; and a 0^{th} monitoring site represents an upstream boundary of the river, namely C₀ is a daily averaged chloride concentration from an upstream inflow of the river.

Variations of chloride loads of adjacent upstream and downstream monitoring sites are determined:

An i^{th} reach is the river reach with sewage outfalls if QᵢCᵢ>Qᵢ₋₁Cᵢ₋₁,

where, i∈[1,n], Cᵢ is a daily averaged chloride concentration of an i^{th} monitoring site; Cᵢ₋₁ is a daily averaged chloride concentration of an upstream i−1^{th} monitoring site; Qᵢ is a daily flow of the i^{th} monitoring site; Qᵢ₋₁ is a daily flow of the upstream i−1^{th} monitoring site; and the 0^{th} monitoring site represents an upstream boundary of the river, namely C₀ is a daily averaged chloride concentration from an upstream inflow of the river, and Q₀ is a daily flow from the upstream inflow of the river.

Second Case:

In a case where there is a tributary in a reach, a river reach with sewage outfalls is determined as follows:

A chloride concentration of each of an upstream monitoring site, the tributary and a downstream monitoring site is compared:

An i^{th} reach is the river reach with sewage outfalls if Cᵢ>max(Cᵢ₋₁,C_{Ti}),

where, i∈[1,n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; and a 0^{th} monitoring site represents an upstream boundary of the river, namely C₀ is a daily averaged chloride concentration from an upstream inflow of the river.

Variations of chloride loads of adjacent upstream and downstream monitoring sites are determined:

An i^{th} reach is the river reach with sewage outfalls if QᵢCᵢ>Qᵢ₋₁Cᵢ₋₁+Q_{Ti}C_{Ti},

where, i∈[1,n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; a 0^{th} monitoring site represents an upstream boundary of the river, namely C₀ is a daily averaged chloride concentration from an upstream inflow of the river; Qᵢ is a daily flow of the i^{th} monitoring site; Qᵢ₋₁ is a daily flow of the upstream i−1^{th} monitoring site; Q_{Ti} is a daily flow that the tributary flows into the i reach; and the 0^{th} monitoring site represents the upstream boundary of the river, namely the C₀ is the daily averaged chloride concentration from the upstream inflow of the river, and Q₀ is a daily flow from the upstream inflow of the river.

A grid-based source-tracing system for sewage outfalls includes a data acquisition device, a data processing device, and a display device.

The data acquisition device is configured to acquire tributary confluence data of a river, monitoring data of monitoring sites, and intensive monitoring data of a river reach with sewage outfalls.

The data acquisition device is an online water level and conductivity monitoring device.

The data processing device is configured to divide reaches according to the tributary confluence data of the river; calculate soft measurement data according to the monitoring data; determine the river reach with sewage outfalls according to the soft measurement data; and analyze the intensive monitoring data of the river reach with sewage outfalls to determine a position of a sewage outfall.

The data processing device in the embodiment is a central processor.

The display device is configured to display the river reach with sewage outfalls and the position of the sewage outfall.

The display device in the embodiment is a display screen.

A computer storage medium stores a computer program thereon, where when executed by a processor, the program implements steps of the grid-based source-tracing method for sewage outfalls.

### Embodiment 2

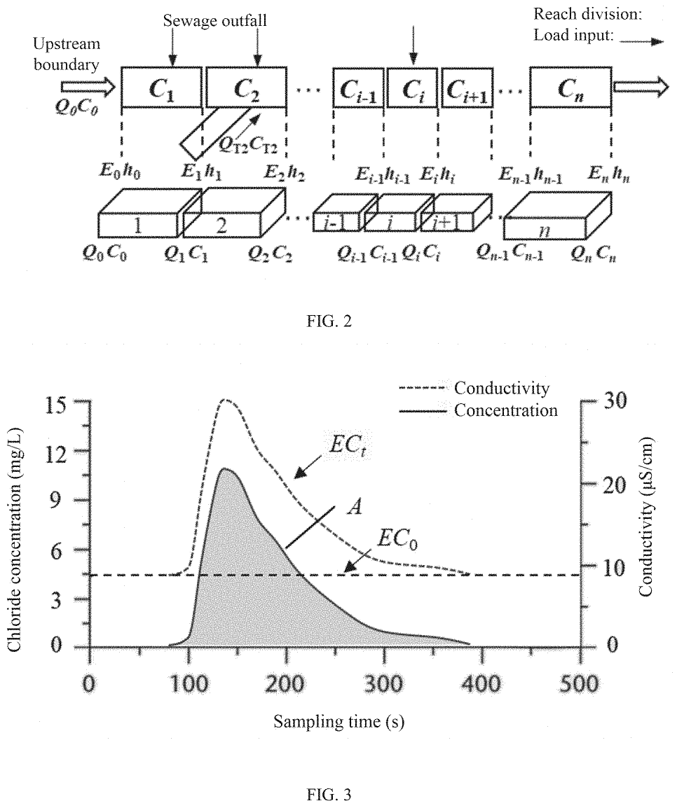

S1: An urban river as shown in FIG. 2 is divided into three reaches according to tributary confluence data, a second reach including a tributary, and an online conductivity and water level monitor is provided at each of positions for dividing the reaches and a confluence of the tributary to synchronously acquire conductivity and water level data for each of monitoring sites.

S2: A chloride-conductivity soft measurement method is constructed.

S21: A chloride as a conservative substance is selected as a water quality monitoring indicator.

S22: Water samples are acquired in the dry weather, monitoring sections being consistent with the positions for dividing the reaches. The samples are continuously acquired once every 2 hours for 2 days. For each sampling point, it is required to acquire water samples at 0.5 m below the water surface. Following the acquisition of the water samples every day, they are sent to laboratories immediately to measure the conductivities and the chloride concentrations.

Measurement on conductivity: the conductivity is measured with a DDS-307 conductivity meter, and then converted into a value at 25° C. through the temperature compensation function.

Measurement on chloride concentration: a silver nitrate titration method (GB 11896-89) is used. In case of a high chloride content, water samples can be diluted with water for measurement.

S23: With a chloride concentration as ay axis and a conductivity as an x axis, linear fitting is performed on the monitoring data with a least-squares method to obtain a chloride concentration-conductivity curve, as shown in FIG. 3.

S3: A water level-flow soft measurement method is constructed.

S31: The flow and the water level are synchronously monitored once every 4 hours for 2 days.

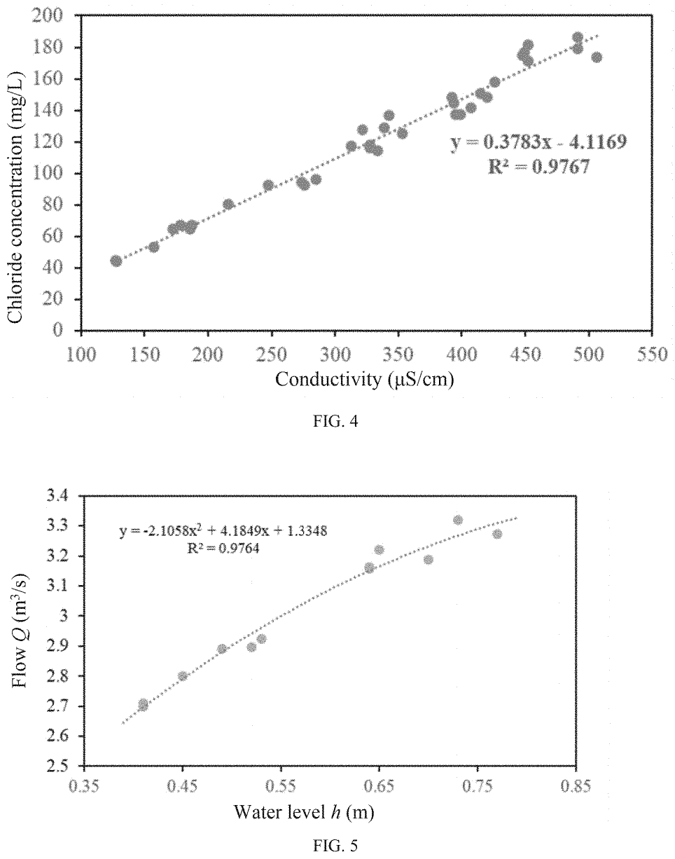

S32: The flow is monitored with a tracer-dilution method: NaCl was selected as a tracer; 5 kg of a NaCl solution was instantly injected into an upstream section of the monitoring site, water samples were continuously taken for 500 s at a fixed interval of 20 s before the NaCl reached the monitoring site, and conductivities of the water samples were measured. The conductivities of the water samples are converted into the chloride concentrations to obtain a time-varying curve of chlorides at the monitoring site, as shown in FIG. 4. According to the chemical mass balance of the chlorides, the flow of the monitoring section is calculated by:

$$Q = \frac{M}{\sum ( {EC}_{t} - {EC}_{0} ) \cdot CF}$$

where, ECₜ is a conductivity when t=t, EC₀ is a background conductivity of the river, M is a mass of injected chloride at upstream site, and CF is a conversion coefficient between the conductivity and the chloride concentration. The CF was 0.38 in the embodiment,

S33: With a flow as an x axis and a water level as a y axis, polynomial fitting is performed on the monitoring data with the least-squares method to obtain the flow-water level curve, as shown in FIG. 5.

S4. A river reach with sewage outfalls is determined.

S41: The online conductivity monitoring data of each of the monitoring sites is converted into a chloride concentration according to the chloride concentration-conductivity curve to obtain a time-varying curve for each of the monitoring sites, thereby obtaining a daily averaged chloride concentration.

By monitoring, the daily averaged conductivities are as follows: E₀ is 232 μS/cm, E₁ is 246 μS/cm, E₂ is 263 μS/cm, E₃ is 260 μS/cm, and E_{T2} is 329 μS/cm. Therefore, the daily averaged chloride concentrations at the monitoring sites are calculated as follows: is 83.6 mg/L, C₁ is 91.7 mg/L, C₂ is 95.9 mg/L, C₃ is 95.8 mg/L, and C_{T2} is 118.8 mg/L.

S42: The online water level monitoring data of each of the monitoring sites is converted into a flow value according to the flow-water level curve to obtain a time-varying curve for each of the monitoring sites, thereby obtaining daily averaged water flow data.

By monitoring, the daily averaged water levels are as follows: h₀ is 0.68 m, h₁ is 0.72 m, h₂ is 0.79 m, h₃ is 0.81 m, and h_{T2} is 0.86 m.

Therefore, the daily averaged water flows at the monitoring sites are calculated as follows: Q₀ is 2.77×105 m³/d, Q₁ is 2.79×105 m³/d, Q₂ is 2.94×105 m³/d, Q₃ is 2.95×105 m³/d, and Q_{T2} is 9.88×103 m³/d.

S403: The river reach with sewage outfalls is determined according to variations of chloride concentrations and chloride loads of upstream and downstream monitoring sites on the basis of the above monitoring data.

Variations of chloride concentrations of adjacent upstream and downstream monitoring sites are determined.

Due to C₁>C₀, the first reach is the river reach with sewage outfalls.

Due to C₁<max(C₂,C_{T2}) and C₂<C₁, whether the second and third reaches are the river reach with sewage outfalls need to be further determined.

Variations of chloride loads of adjacent upstream and downstream monitoring sites are determined:

Due to Q₂C₂>Q₁C₁+Q_{T2}C_{T2}, the second reach is the river reach with sewage outfalls.

Due to Q₃C₃>Q₂C₂, the third reach is the river reach with sewage outfalls.

S5: Since the three reaches are the river reach with sewage outfalls, conductivity and water level monitoring sites are intensively arranged based on a dichotomizing theory to further divide the three reaches into six reaches. Likewise, whether the reaches are the river reach with sewage outfalls is determined respectively according to the pollutant source tracing method in the present disclosure.

Specifically, with the first reach for example, if C₁−C₀>0, it is indicated that the first reach is the river reach with sewage outfalls, and the chloride concentration of the sewage is higher than the background value for the chloride concentration of the river. By monitoring the middle of the first reach, the daily averaged conductivity of the section is 233 μS/cm, and the daily averaged chloride concentration C₁₂ is calculated as 84.0 mg/L, thus determining that the key sewage outfall is located in the latter half of the first reach. To further narrow the investigation range of the sewage outfall, the latter half of the first reach can be dichotomized to implement source tracing for the sewage outfall of the river.

Each embodiment of the present disclosure is described in a progressive manner, each embodiment focuses on the difference from other embodiments, and the same and similar parts between the embodiments may refer to each other. Since a device disclosed in the embodiments corresponds to a method disclosed in the embodiments, its description is relatively simple, and reference may be made to partial description of the method for relevant contents.

The above description of the disclosed embodiments enables those skilled in the art to achieve or use the present disclosure. Various modifications to these embodiments are readily apparent to those skilled in the art, and the generic principles defined herein may be practiced in other embodiments without departing from the spirit or scope of the present disclosure. Thus, the present disclosure is not limited to the embodiments shown herein but falls within the widest scope consistent with the principles and novel features disclosed herein.

What is claimed is:

1. A grid-based source-tracing method for sewage outfalls, specifically comprising the following steps:
dividing reaches: dividing a river into a plurality of reaches to obtain divided reaches;
determining monitoring sites: determining the monitoring sites according to the divided reaches;
acquiring soft measurement data: acquiring monitoring data of each of the monitoring sites, and calculating the soft measurement data;
determining a river reach with sewage outfalls: determining the river reach with sewage outfalls according to upstream and downstream soft measurement data; and
obtaining a position of a sewage outfall: intensively arranging monitoring sites in the river reach with sewage outfalls to subdivide the river reach with sewage outfalls, thereby determining the position of the sewage outfall.

2. The grid-based source-tracing method according to claim 1, wherein when the monitoring sites are determined, a position for dividing the plurality of reaches and a confluence of a tributary are determined as the monitoring sites.

3. The grid-based source-tracing method according to claim 1, wherein the step of acquiring the monitoring data comprises:
S31: acquiring a conductivity of each of the monitoring sites, and obtaining a chloride concentration of each of the monitoring sites according to a chloride concentration-conductivity curve; and
S32: synchronously acquiring a water level of each of the monitoring sites, and obtaining a flow of each of the monitoring sites according to a flow-water level curve.

4. The grid-based source-tracing method according to claim 3, wherein the chloride concentration-conductivity curve is drawn as follows:
S311: acquiring a water sample from a fixed depth of each of the monitoring sites at a fixed frequency within a fixed time;
S312: measuring a conductivity and a chloride concentration of the water sample; and
S313: performing a fitting on the conductivity and the chloride concentration with a least-squares method to obtain the chloride concentration-conductivity curve with the chloride concentration as ay axis and the conductivity as an x axis.

5. The grid-based source-tracing method according to claim 3, wherein the flow-water level curve is drawn as follows:
S321: synchronously acquiring a flow and a water level of each of the monitoring sites at a fixed frequency within a fixed time; and
S322: performing polynomial fitting on the flow and the water level of each of the monitoring sites with the least-squares method to obtain the flow-water level curve with the flow as an x axis and the water level as ay axis.

6. The grid-based source-tracing method according to claim 3, wherein the river reach with sewage outfalls is determined according to the upstream and downstream soft measurement data of the monitoring sites, wherein there are two cases, comprising a first case where the reach comprises a tributary and a second case where the reach does not comprise a tributary.

7. The grid-based source-tracing method according to claim 6, wherein in the second case where the reach does not comprise the tributary, the river reach with sewage outfalls is determined as follows:
determining variations of chloride concentrations of adjacent upstream and downstream monitoring sites:
determining, if Cᵢ>Cᵢ₋₁, that an i^{th} reach is the river reach with sewage outfalls,
wherein, i∈[1,n], Cᵢ is a daily averaged chloride concentration of an i^{th} monitoring site; Cᵢ₋₁ is a daily averaged chloride concentration of an upstream i−1^{th} monitoring site; and a 0^{th} monitoring site represents an upstream boundary of the river, indicating that C₀ is a daily averaged chloride concentration from an upstream inflow of the river; and
determining variations of chloride loads of the adjacent upstream and downstream monitoring sites:
determining, if QᵢCᵢ>Qᵢ₋₁Cᵢ₋₁, that the i^{th} reach is the river reach with sewage outfalls,
wherein, i∈[1,n], Cᵢ is the daily averaged chloride concentration of the monitoring site; Cᵢ₋₁ is the daily averaged chloride concentration of the upstream i−1^{th} monitoring site; Qᵢ is a daily flow of the i^{th} monitoring site; Qᵢ₋₁ is a daily flow of the upstream i−1^{th} monitoring site; and the 0^{th} monitoring site represents an upstream boundary of the river, indicating that C₀ is the daily averaged chloride concentration from the upstream inflow of the river, and Q₀ is a daily flow from the upstream inflow of the river.

8. The grid-based source-tracing method according to claim 6, wherein in the first case where the reach comprises the tributary, the river reach with sewage outfalls is determined as follows:
comparing a chloride concentration of each of an upstream monitoring site, the tributary and a downstream monitoring site:
determining, if Cᵢ>max(Cᵢ₋₁,C_{Ti}), that an i^{th} reach is the river reach with sewage outfalls,
wherein, i∈[=1,n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; and a 0^{th} monitoring site represents an upstream boundary of the river, indicating that C₀ is a daily averaged chloride concentration from an upstream inflow of the river; and
determining variations of chloride loads of the adjacent upstream and downstream monitoring sites:
determining, if QᵢCᵢ>Qᵢ₋₁Cᵢ₋₁+Q_{Ti}C_{Ti}, that the i^{th} reach is the river reach with sewage outfalls,
wherein, i∈[1,n], Cᵢ is the daily averaged chloride concentration of the i^{th} monitoring site; Cᵢ₋₁ is the daily averaged chloride concentration of the upstream i^{th} monitoring site; C_{Ti} is the daily averaged chloride concentration of the tributary converges into the i^{th} reach; the 0^{th} monitoring site represents the upstream boundary of the river, indicating that C₀ is a daily averaged chloride concentration from the upstream inflow of the river; is a daily flow of the i^{th} monitoring site; Qᵢ₋₁ is a daily flow of the upstream i−1^{th} monitoring site; Q_{Ti} is a daily flow of the tributary that converges into the i reach; and the 0^{th} monitoring site represents the upstream boundary of the river, indicating that the C₀ is the daily averaged chloride concentration from the upstream inflow of the river, and Q₀ is a daily flow from the upstream inflow of the river.

9. A grid-based source-tracing system for sewage outfalls, comprising a data acquisition device, a data processing device, and a display device, wherein
the data acquisition device is configured to acquire tributary confluence data of a river, monitoring data of monitoring sites, and intensive monitoring data of a river reach with sewage outfalls;
the data processing device is configured to divide reaches according to the tributary confluence data of the river, calculate soft measurement data according to the monitoring data; determine the river reach with sewage outfalls according to the soft measurement data, and analyze the intensive monitoring data of the river reach with sewage outfalls to determine a position of a sewage outfall; and
the display device is configured to display the river reach with sewage outfalls.

10. A computer-readable storage medium, storing a computer program thereon, wherein when executed by a processor, the computer program implements steps of the grid-based source-tracing method according to claim 1.

11. The computer-readable storage medium according to claim 10, wherein when the monitoring sites are determined, a position for dividing the plurality of reaches and a confluence of a tributary are determined as the monitoring sites.

12. The computer-readable storage medium according to claim 10, wherein the step of acquiring the monitoring data comprises:
S31: acquiring a conductivity of each of the monitoring sites, and obtaining a chloride concentration of each of the monitoring sites according to a chloride concentration-conductivity curve; and
S32: synchronously acquiring a water level of each of the monitoring sites, and obtaining a flow of each of the monitoring sites according to a flow-water level curve.

13. The computer-readable storage medium according to claim 12, wherein the chloride concentration-conductivity curve is drawn as follows:
S311: acquiring a water sample from a fixed depth of each of the monitoring sites at a fixed frequency within a fixed time;
S312: measuring a conductivity and a chloride concentration of the water sample; and
S313: performing a fitting on the conductivity and the chloride concentration with a least-squares method to obtain the chloride concentration-conductivity curve with the chloride concentration as ay axis and the conductivity as an x axis.

14. The computer-readable storage medium according to claim 12, wherein the flow-water level curve is drawn as follows:
S321: synchronously acquiring a flow and a water level of each of the monitoring sites at a fixed frequency within a fixed time; and
S322: performing polynomial fitting on the flow and the water level of each of the monitoring sites with the least-squares method to obtain the flow-water level curve with the flow as an x axis and the water level as ay axis.

15. The computer-readable storage medium according to claim 12, wherein the river reach with sewage outfalls is determined according to the upstream and downstream soft measurement data of the monitoring sites, wherein there are two cases, comprising a first case where the reach comprises a tributary and a second case where the reach does not comprise a tributary.

16. The computer-readable storage medium according to claim 15, wherein in the second case where the reach does not comprise the tributary, the river reach with sewage outfalls is determined as follows:
determining variations of chloride concentrations of adjacent upstream and downstream monitoring sites:
determining, if Cᵢ>Cᵢ₋₁, that an i^{th} reach is the river reach with sewage outfalls,
wherein, i∈[1,n], Cᵢ is a daily averaged chloride concentration of an i^{th} monitoring site; Cᵢ₋₁ is a daily averaged chloride concentration of an upstream i−1^{th} monitoring site; and a 0^{th} monitoring site represents an upstream boundary of the river, indicating that C₀ is a daily averaged chloride concentration from an upstream inflow of the river; and
determining variations of chloride loads of the adjacent upstream and downstream monitoring sites:
determining, if QᵢCᵢ>Qᵢ₋₁Cᵢ₋₁, that the i^{th} reach is the river reach with sewage outfalls,
wherein, i∈[1,n], Cᵢ is the daily averaged chloride concentration of the i^{th} monitoring site; Cᵢ₋₁ is the daily averaged chloride concentration of the upstream i−1^{th} monitoring site; Qᵢ is a daily flow of the i^{th} monitoring site; Qᵢ₋₁ is a daily flow of the upstream i−1^{th} monitoring site; and the 0^{th} monitoring site represents an upstream boundary of the river, indicating that C₀ is the daily averaged chloride concentration from the upstream inflow of the river, and Q₀ is a daily flow from the upstream inflow of the river.

17. The computer-readable storage medium according to claim 15, wherein in the first case where the reach comprises the tributary, the river reach with sewage outfalls is determined as follows:
comparing a chloride concentration of each of an upstream monitoring site, the tributary and a downstream monitoring site:
determining, if Cᵢ>max(Cᵢ₋₁,C_{Ti}), that an i^{th} reach is the river reach with sewage outfalls,
wherein, i∈[1n], Cᵢ is a daily averaged chloride concentration of an i^{th} monitoring site; Cᵢ₋₁ is a daily averaged chloride concentration of an upstream i−1^{th} monitoring site; C_{Ti} is a daily averaged chloride concentration of the tributary converges into the i^{th} reach; and a 0^{th} monitoring site represents an upstream boundary of the river, indicating that C₀ is a daily averaged chloride concentration from an upstream inflow of the river; and
determining variations of chloride loads of the adjacent upstream and downstream monitoring sites:
determining, if QᵢCᵢ>Qᵢ₋₁Cᵢ₋₁+Q_{Ti}C_{Ti}, that the i^{th} reach is the river reach with sewage outfalls,
wherein, i∈[1,n], Cᵢ is the daily averaged chloride concentration of the i^{th} monitoring site; Cᵢ₋₁ is the daily averaged chloride concentration of the upstream i−1^{th} monitoring site; the 0^{th} monitoring site represents the upstream boundary of the river, indicating that i^{th} is a daily averaged chloride concentration from the upstream inflow; Qᵢ is a daily flow of the i^{th} monitoring site; Qᵢ₋₁ is a daily flow of the upstream i−1^{th} monitoring site; Q_{Ti} is a daily flow of the tributary that converges into the i reach; and the 0^{th} monitoring site represents the upstream boundary of the river, indicating that the C₀ is the daily averaged chloride concentration from the upstream inflow of the river, and Q₀ is a daily flow from the upstream inflow of the river.
